(19) United States
(12) Patent Application Publication
(10) Pub. No.: US 2005/0241961 A1
(43) Pub. Date: Nov. 3, 2005

(54) Synthetic suture card

(76) Inventor: Patrick J. Ferguson, Portland, OR (US)

Correspondence Address: GLENN C. BROWN, PC, 777 NW WALL STREET, SUITE 308, BEND, OR, 97701, US

(21) Appl. No.: 10/835,870

(22) Filed: Apr. 29, 2004

(51) Int. Cl. A61B 17/06

(52) U.S. Cl. 206/63.3; 206/574

(57) ABSTRACT

A shipping and storage container for surgical sutures, with or without an attached needle comprising a non-cellulosic, hydrophobic material folded to define a container, the container defining a first cavity adapted for receiving and encasing a suture material. The non-cellulosic, hydrophobic material is preferably a polymeric material such as polypropylene.

This application is related to suture cards in which surgical sutures are packaged for distribution and storage.

Surgical sutures are precut to predetermined lengths, attached to a needle, and sealed in a clear plastic wrapper. In some instances, one or several of the packaged sutures are then packaged in a folded container made of paper or other cellulosic material. In other instances the suture and attached needle are placed directly into the card without first being placed in a clear plastic wrapper. The container, or card, is imprinted with identifying and regulatory information such as the brand name, the manufacturer, the type of suture material, the suture length, the attached needle shape and size, lot number, and any other information deemed useful or necessary by the manufacturer or distributor. The suture and card are then sterilized, and distributed to distributors and eventually to end users.

During distribution, however, the cards (and sutures if not separately packaged) are exposed to ambient, non-sterile conditions, and become contaminated to a small yet significant degree by molds and fungus and other contaminants on the card. Contaminants on the cards can adversely impact the sterility of the storage areas and other spaces in a hospital or office storage area.

A need therefore exists for an improved suture card that can be manufactured, labeled and distributed by existing methods and channels, but which at the same time is resistant to the uptake of ambient moisture during distribution and storage.

### SUMMARY OF THE INVENTION

Applicant has discovered that the accumulation and proliferation of molds, fungus and contaminants on known suture cards is promoted by the uptake of minute amounts of moisture in the paper or cellulosic cards. Even small amounts of moisture on and in the interstices of the cellulosic suture card can lead to significantly higher contaminant levels, and the resulting degradation of the sterile conditions in storage areas and operating rooms.

Applicant has discovered that by the judicious selection of a non-cellulosic material of construction, a suture card can represent a significantly lower contaminant loading source in these sensitive areas of medical facilities. In one preferred embodiment of the invention, an improved suture card is formed of a synthetic paper that can be characterized as a mineral-reinforced polypropylene printing and converting film material. The film material can be printed, folded and sterilized by using known equipment and techniques and thereby requires little if any capital investment to be integrated into existing manufacturing processes. At the same time, it exhibits the required levels of strength and rigidity to meet the requirements of suture packaging.

These and other aspects of the invention will be described in greater detail by reference to the drawings and detailed description.

### BRIEF DESCRIPTION OF THE DRAWINGS

FIG. 1 is a top plan view of a typical folded suture card.

FIG. 2 is a partially opened suture card of the type shown in FIG. 1, and showing the needle and a first portion of the suture exposed and ready to be removed from the suture card, and showing the remaining portion of the suture in phantom.

FIG. 3 is a partially opened suture card as shown in FIG. 2, and showing the needle and a first portion of the suture removed from the card and ready to use.

FIG. 4 is a top plan view of a die-cut suture card blank before being folded into the configuration shown in FIG. 1.

FIG. 5 is a bottom plan view of the fully folded suture card shown in FIG. 1 and formed from the die-cut suture card blank shown in FIG. 4.

### DETAILED DESCRIPTION

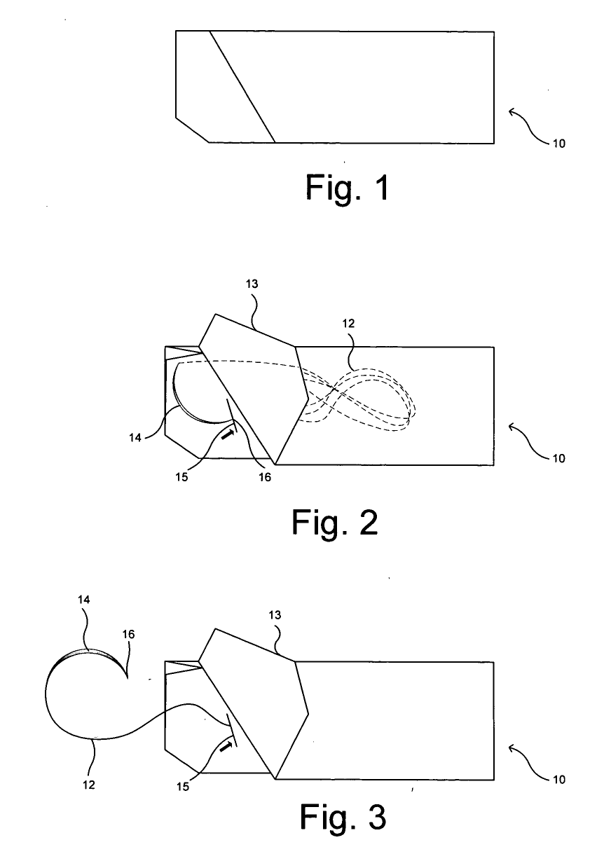

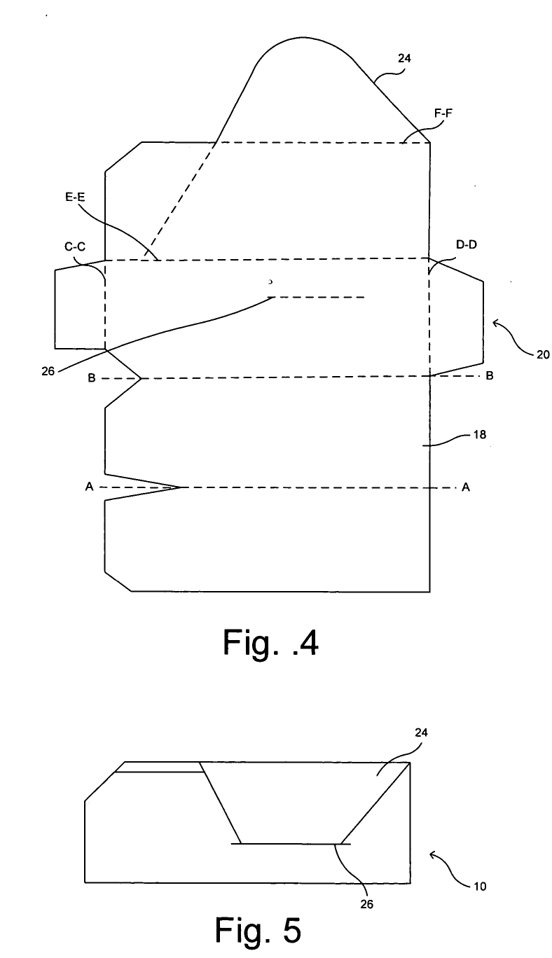

Referring to FIGS. 1-4, a suture card is shown generally at 10 with a suture 12 and attached needle 14 contained within. The tip of needle 14 is stowed in slot 15 as best seen in FIG. 2, with suture 12 coiled within the folded suture card as shown in phantom in FIG. 2. When the needle and suture are needed, flap 13 is folded back to reveal and present needle 14 as also shown in FIG. 2, which along with suture 12 can then be removed from the suture card.

Referring now to FIG. 3, suture card 12 is formed by folding a die-cut blank 20 along a series of predetermined fold lines to form the card. The suture 12 and needle 14 are first placed at 18, and the blank is folded sequentially along lines A-A, B-B, C-C, and D-D. The point 16 of needle 14 is then placed in slot 15 as described above with reference to FIG. 1. The partially formed suture card is then folded sequentially along lines E-E, and F-F. Tab 24 is then placed in slot 26 to secure the suture card in its folded position until ready for use. The suture card is then shipped and stored in that configuration until the suture is used. At that time, flap 16 is folded back as shown in FIG. 2, revealing needle 14 in its stored position. Needle 14 is removed from slot 22 and suture 12 is pulled from the suture card as needed.

There are numerous variations possible in the shape of the die-cut blank and the finished suture card, and the invention is not intended to be limited to the embodiment described above. It is also possible to include one or more prepackaged suture/needle assemblies within a suture card, rather than the self-dispensing suture card described above.

Whatever the particular configuration of the suture card, applicant has discovered that the ongoing problem of contamination of the suture card and/or its contents can be primarily attributed to moisture uptake in the cellulosic material from which known suture cards are formed. Applicant has discovered that the problem can be reduced and in some cases alleviated by the making the suture card from a hydrophobic polymeric material. Applicant has determined that significant contaminant reductions can be achieved with materials that demonstrate a moisture uptake of no more than preferably 8% by weight, more preferably no more than 7% by weight, and most preferably no more than 5% by weight. The material must also, however, embody surface properties that permit the necessary information to be printed permanently and attractively on the suture card, that exhibits the necessary strength, and that can be die cut and folded using existing processing equipment.

Applicant has discovered that one particularly well-suited material for use in connection with the invention is an opaque, mineral-reinforced polypropylene sheet material. While the particular material is known to be a suitable printing stock for cards, tags, and the like, the material's value as a material for forming suture cards was not apparent until applicant discovered the role of absorbed ambient moisture in promoting contamination of the suture cards during shipment and storage.

Armed with this discovery, applicant has now identified this material as the preferred material for the manufacture of hydrophobic suture cards. Applicant has identified a commercial source for one such material. It is available from Granwell Products, Inc of West Caldwell, N.J., which offers the material under as “Polylith®” Synthetic Paper, Product Code GC-3. The preferred material is either 4 or 6 mils in thickness with an opacity of about 85-87%, a whiteness of 85-88 W, a tensile strength of about 3000 psi, and a tear strength of between 2100 and 2500 gm/mm.

While applicant has identified this particular material as the preferred material, the invention is not intended to be limited to this particular material. It is possible and even likely that other hydrophobic materials of which applicant is not now aware could also embody the necessary combination of additional qualities to render them suitable for use according to the invention. Those of skill in the art will recognize that the invention can depart from the description of the preferred embodiment in these and other ways without departing from the scope of the claims.

1. A shipping and storage container for surgical sutures, with or without an attached needle, comprising a non-cellulosic, hydrophobic material folded to define a container, the container defining a first cavity adapted for receiving and encasing a suture material.

2. A shipping and storage container for surgical sutures according to claim 1 wherein the non-cellulosic, hydrophobic material comprises a polymeric material.

3. A shipping and storage container for surgical sutures according to claim 1 further comprising the non-cellulosic, hydrophobic material comprising a non-woven material.

4. A shipping and storage container for surgical sutures according to claim 1 wherein the non-cellulosic, hydrophobic material comprises polyproplylene.

5. A shipping and storage container for surgical sutures according to claim 1 wherein the non-cellulosic, hydrophobic material exhibits an ambient moisture uptake of no more than 5% by weight.

6. A shipping and storage container for surgical sutures according to claim 1 wherein the non-cellulosic, hydrophobic material exhibits an ambient moisture uptake of no more than 7% by weight.

7. A shipping and storage container for surgical sutures according to claim 1 wherein the non-cellulosic, hydrophobic material exhibits an ambient moisture uptake of no more than 8% by weight.

8. A shipping and storage container for surgical sutures wherein the container defines a second cavity adapted for receiving a needle.
